(19) United States
(12) Patent Application Publication
(10) Pub. No.: US 2021/0263052 A1
(43) Pub. Date: Aug. 26, 2021

(54) BLOOD COAGULATION SYSTEM ANALYSIS DEVICE

(71) Applicant: Sony Corporation, Tokyo (JP)

(72) Inventors: Yoshihito Hayashi, Chiba (JP); Tokujiro Uchida, Kanagawa (JP); Yudai Yamamoto, Tokyo (JP)

(73) Assignee: Sony Corporation, Tokyo (JP)

(21) Appl. No.: 17/260,969

(22) PCT Filed: Jun. 12, 2019
(86) PCT No.: PCT/JP2019/023191

§ 371 Date: Jan. 15, 2021

(30) Foreign Application Priority Data
Jul. 25, 2018 (JP) 2018-139300

(51) Int. Cl. G01N 33/86; G01N 27/22

(52) U.S. Cl. CPC G01N 33/86; G01N 27/228

(57) ABSTRACT

To provide a blood coagulation system analysis device capable of easily and rapidly evaluating a human tissue factor pathway inhibitor.

A blood coagulation system analysis device provided with a pair of electrodes, an application unit that applies an alternating voltage to the pair of electrodes at a predetermined time interval, a measurement unit that measures complex permittivity of a blood sample arranged between the pair of electrodes, and an analysis unit that evaluates a human tissue factor pathway inhibitor (TFPI) on the basis of the complex permittivity at a specific frequency in a predetermined period measured at the time interval after anticoagulant action acting on the blood sample is released.

### TECHNICAL FIELD

The present technology relates to a blood coagulation system analysis device.

### BACKGROUND ART

Conventionally, there is a blood coagulation test as a clinical method of analyzing a blood condition. As a general blood coagulation test, a blood coagulation test represented by prothrombin time (PT) and activated partial thromboplastin time (APTT) is known. These methods are methods of analyzing coagulation reactivity by proteins involved in coagulation reaction contained in plasma obtained by centrifuging a blood sample.

However, although the above-described test method is suitable for evaluating a significant decrease in blood coagulation ability, that is, a tendency to bleed, this does not suitable for capturing a significant increase in blood coagulation ability, that is, a thrombotic tendency, or a subtle change in blood coagulation ability, and it is also difficult to evaluate a human tissue factor pathway inhibitor (hereinafter, also simply referred to as “TFPI”) in blood.

The TFPI is one of central molecules in charge of an adjusting mechanism of a blood coagulation system, and when a blood concentration thereof increases, there is a possibility that, even in a blood vessel damaged site where blood coagulation reaction should originally occur, the reaction is inhibited, and effective hemostasis cannot be performed. Furthermore, blood TFPI cannot be neutralized by protamine and the like, and an unexpected blood coagulation inhibitory state continues, which is one of causes such as continuous postoperative bleeding. In contrast, it is not easy to determine whether or not the blood TFPI is the cause in each case because there is a plurality of other factors that keeps the blood coagulation inhibitory state. Therefore, there is a clear need in the medical field to rapidly and easily evaluate the TFPI concentration in the blood and TFPI activity.

Here, as another functional test, there are thromboelastography and thromboelastometry, which are commercialized as TEG (registered trademark) and ROTEM (registered trademark), respectively, but there are reasons such as (1) the measurement is not automated and the test result depends on the procedure of the measurer, (2) this is susceptible to vibration, (3) the quality control (QC) procedure is complicated, and the reagent for QC is expensive, and (4) the interpretation of the output signal (thromboelastogram) requires proficient skills, so that this is not sufficiently popularized. Furthermore, the sensitivity thereof to deficiency and inhibitory effects of each coagulation factor of the extrinsic system and intrinsic system is not so high, so that there is a possibility that this cannot satisfy the needs of the medical field.

On the other hand, in recent years, as another method capable of easily and accurately evaluating blood coagulation measurement, a method of performing dielectric measurement of the blood coagulation process has been devised (for example, Patent Documents 1 and 2). In this method, a capacitor-type sample unit including a pair of electrode pairs is filled with a blood sample and an alternating electric field is applied thereto to measure a change in complex permittivity accompanying a coagulation process of the blood sample. Non-Patent Document 1 discloses that a process of coagulation and fibrinolytic response may be easily monitored by using this method. However, no knowledge has yet been obtained regarding the evaluation of the TFPI.

### CITATION LIST

### Patent Document

Patent Document 1: Japanese Patent Application Laid-Open No. 2010-181400
Patent Document 2: Japanese Patent Application Laid-Open No. 2012-194087

### Non-Patent Document

Non-Patent Document 1: Y. Hayashi et al., Analytical Chemistry 87 (19), 10072-10079 (2015)

### SUMMARY OF THE INVENTION

### Problems to be Solved by the Invention

As described above, despite the need in the medical field to evaluate the TFPI, there is currently no choice but to analyze plasma components obtained by centrifugation, which takes time and effort, so that this is not performed in perioperative clinical examinations.

Therefore, a principal object of the present technology is to provide a blood coagulation system analysis device capable of easily and rapidly evaluating a human tissue factor pathway inhibitor.

### Solutions to Problems

The present technology provides a blood coagulation system analysis device provided with a pair of electrodes, an application unit that applies an alternating voltage to the pair of electrodes at a predetermined time interval, a measurement unit that measures complex permittivity of a blood sample arranged between the pair of electrodes, and an analysis unit that evaluates a human tissue factor pathway inhibitor (TFPI) on the basis of the complex permittivity at a specific frequency in a predetermined period measured at the time interval after anticoagulant action acting on the blood sample is released.

In the present technology, the TFPI may be evaluated by using a tissue factor and an anti-TFPI antibody. In this case, the analysis unit may evaluate the TFPI on the basis of the complex permittivity measured by using the tissue factor and the anti-TFPI antibody and the complex permittivity measured by using the tissue factor.

Furthermore, in the present technology, a heparin decomposing agent and/or a heparin neutralizing agent may further be used. In this case, the analysis unit may evaluate the TFPI on the basis of the complex permittivity measured by using the tissue factor, the heparin decomposing agent and/or the heparin neutralizing agent, and the anti-TFPI antibody, and the complex permittivity measured by using the tissue factor and the heparin decomposing agent and/or the heparin neutralizing agent.

Moreover, in the present technology, a feature amount extracted from a complex permittivity spectrum at the specific frequency may be used at the time of the evaluation. In this case, the feature amount may be a time feature amount and/or a gradient feature amount extracted from the complex permittivity spectrum at the specific frequency. In this case, the gradient feature amount may be extracted on the basis of the time feature amount extracted from the complex permittivity spectrum at the specific frequency. Furthermore, in this case, the feature amount may be any one or more selected from a group including time CT0 at which a local maximum value of the complex permittivity is given at a low frequency of 100 kHz or higher and lower than 3 MHz, time CT1 at which a maximum gradient is given at the low frequency, a maximum gradient CFR at the low frequency, time CT4 when an absolute value of the gradient reaches a predetermined percentage of the CFR after the CT1, time CT at which a local minimum value of the complex permittivity is given at a high frequency of 3 to 30 MHz, time CT3 at which a maximum gradient is given at the high frequency, a maximum gradient CFR2 at the high frequency, time CT2 at which an absolute minimum value of the complex permittivity is given when a straight line is drawn at the gradient of CFR2 from CT3 after the CT and before the CT3, and time CT5 when an absolute value of the gradient reaches a predetermined percentage of the CFR2 after the CT3.

In addition, in the present technology, the analysis unit may analyze a degree of postoperative bleeding risk. In this case, the bleeding risk may be a bleeding amount.

Furthermore, the present technology may further be provided with one or a plurality of electrical measurement containers including an assay that at least evaluates extrinsic coagulation ability.

In the present technology, the term “complex permittivity” also includes an amount of electricity equivalent to the complex permittivity. Examples of the amount of electricity equivalent to the complex permittivity include complex impedance, complex admittance, complex capacitance, and complex conductance, which may be converted to each other by a simple electricity amount conversion. Furthermore, the measurement of the “complex permittivity” includes the measurement of only a real part or only an imaginary part. Furthermore, in the present technology, a “blood sample” may be a sample containing erythrocytes and a liquid component such as plasma, and is not limited to the blood itself. More specifically, for example, there is a liquid sample containing a blood component such as whole blood, plasma, or dilution thereof and/or a drug-added substance and the like.

### Effects of the Invention

According to the present technology, a human tissue factor pathway inhibitor may be easily and rapidly evaluated.

Note that the effects herein described are not necessarily limited and may be any of the effects described in the present disclosure.

### BRIEF DESCRIPTION OF DRAWINGS

FIG. 1 is a schematic conceptual diagram schematically illustrating a concept of a blood coagulation system analysis device 100 according to the present technology.

FIG. 2 is a cross-sectional view schematically illustrating an example of an embodiment of an electrical measurement container 101.

FIG. 3 is a drawing-substituting graph for explaining a measurement example of a complex permittivity spectrum (three-dimensional).

FIG. 4 is a drawing-substituting graph for explaining a measurement example of a complex permittivity spectrum (two-dimensional).

FIG. 5 is a drawing-substituting graph illustrating an example of a feature amount extracted from the complex permittivity spectrum.

FIGS. 6A and 6B are drawing-substituting graphs illustrating a relationship between a TFPI concentration in plasma and a bleeding amount within 24 hours after surgery obtained in a measurement group examined this time.

FIG. 7 is a drawing-substituting graph comparing results of EXHNT and EXHN focusing on CT0 from an analysis result of the blood coagulation system analysis device in the measurement group examined this time.

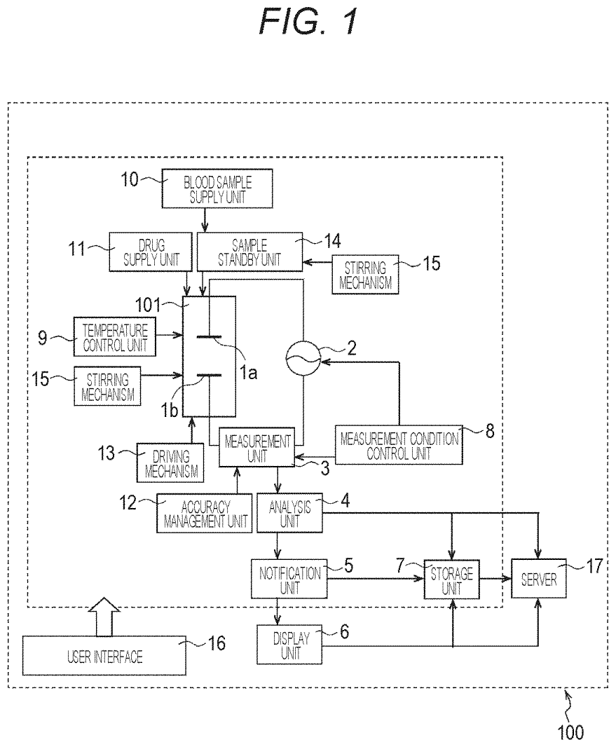

### MODE FOR CARRYING OUT THE INVENTION

Hereinafter, a preferred mode for carrying out the present technology is described with reference to the drawings.

The embodiment hereinafter described illustrates an example of a representative embodiment of the present technology, and the scope of the present technology is not narrowed by them. Note that the description is given in the following order.

1. Blood coagulation system analysis device 100

(1) Pair of electrodes 1a and 1b

(1-1) Electrical measurement container 101

(1-2) Connection unit 102

(1-3) Container holding unit 103

(2) Application unit 2

(3) Measurement unit 3

(4) Analysis unit 4

(5) Notification unit 5

(6) Display unit 6

(7) Storage unit 7

(8) Measurement condition control unit 8

(9) Temperature control unit 9

(10) Blood sample supply unit 10

(11) Drug supply unit 11

(12) Accuracy management unit 12

(13) Driving mechanism 13

(14) Sample standby unit 14

(15) Stirring mechanism 15

(16) User Interface 16

(17) Server 17

(18) Others

1. Blood Coagulation System Analysis Device 100

A blood coagulation system analysis device 100 at least includes a pair of electrodes 1a and 1b, an application unit 2, a measurement unit 3, and an analysis unit 4. Furthermore, the blood coagulation system analysis device 100 may also be provided with other units such as a notification unit 5, a display unit 6, a storage unit 7, a measurement condition control unit 8, a temperature control unit 9, a blood sample supply unit 10, a drug supply unit 11, an accuracy management unit 12, a driving mechanism 13, a sample standby unit 14, a stirring mechanism 15, a user interface 16, and a server 17 as necessary. Hereinafter, each unit is described in detail.

(1) Pair of Electrodes 1a and 1b

The pair of electrodes 1a and 1b come into contact with a blood sample B at the time of measurement and apply a required voltage to the blood sample B.

Arrangement, forms and the like of the pair of electrodes 1a and 1b are not especially limited, and the pair of electrodes 1a and 1b may be freely designed as appropriate as long as the required voltage may be applied to the blood sample B; however, the pair of electrodes 1a and 1b are preferably integrally formed with an electrical measurement container 101 to be described later in the present technology.

A material forming the electrodes 1a and 1b is not especially limited, and one or two or more types of well-known electrically conductive materials may be freely selected to be used as appropriate as long as they do not affect a state and the like of the blood sample B to be analyzed. Specifically, for example, there are titanium, aluminum, stainless, platinum, gold, copper, graphite and the like.

In the present technology, it is preferable to form the electrodes 1a and 1b especially of the electrically conductive material including titanium among them.

Titanium has a property of having low coagulation activity with respect to a blood sample, so that this is suitable for measuring the blood sample B.

(1-1) Electrical Measurement Container 101

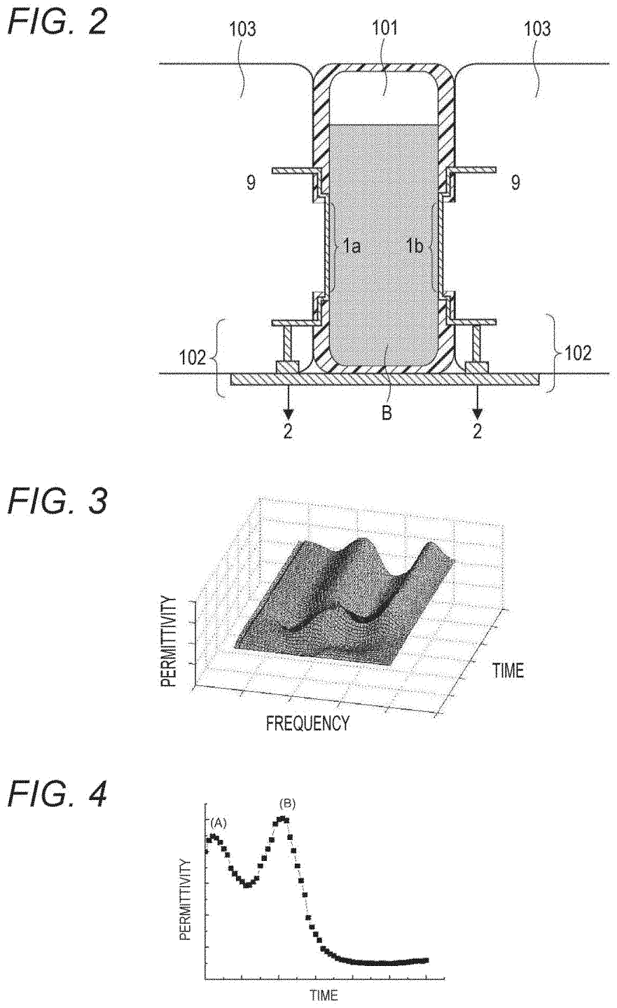

FIG. 2 is a cross-sectional view schematically illustrating an example of an embodiment of the electrical measurement container 101. The electrical measurement container 101 holds the blood sample B to be analyzed. In the blood coagulation system analysis device 100 according to the present technology, the number of the electrical measurement containers 101 is not especially limited, and one or a plurality of electrical measurement containers 101 may be freely arranged as appropriate according to an amount, a type and the like of the blood sample B to be analyzed.

In the blood coagulation system analysis device 100 according to the present technology, complex permittivity is measured in a state in which the blood sample B is held in the electrical measurement container 101. Therefore, the electrical measurement container 101 is preferably configured to be sealable in a state of holding the blood sample B. However, the sealable configuration is not necessarily required if it is possible to hold during time required for measuring the complex permittivity and there is no influence on the measurement.

A specific method of introducing the blood sample B into the electrical measurement container 101 and sealing is not especially limited, and introduction may be performed by a free method as appropriate according to the form and the like of the electrical measurement container 101. For example, there is a method of providing a lid on the electrical measurement container 101, introducing the blood sample B by using a pipette and the like, and thereafter closing the lid to seal and the like.

The form of the electrical measurement container 101 is not especially limited as long as the blood sample B to be analyzed may be held in the device, and may be freely designed as appropriate. Furthermore, the electrical measurement container 101 may include one or a plurality of containers.

A specific form of the electrical measurement container 101 is not especially limited and may be freely designed as appropriate according the state and the like of the blood sample B as long as the blood sample B to be analyzed may be held: a cylinder, a polygonal tubular body having a polygonal cross-section (triangle, quadrangle, or polygon with more angles), a cone, a polygonal pyramid having a polygonal cross-section (triangle, quadrangle, or polygon with more angles), a combination of one or two or more types of them or the like.

Furthermore, a material forming the container 101 is not especially limited, too, and may be freely selected as appropriate as long as this does not affect the state and the like of the blood sample B to be analyzed. In the present technology, it is especially preferable that the container 101 is made by using resin from the viewpoint of ease in processing and shaping. In the present technology, a type of resin which may be used and the like is not especially limited; one or two or more types of resin applicable to holding of the blood sample B may be freely selected to be used as appropriate. For example, there are hydrophobic and insulating polymer such as polypropylene, polymethylmethacrylate, polystyrene, acrylic, polysulfone, and polytetrafluoroethylene, co-polymer, blended polymer and the like.

In the present technology, it is preferable to form the electrical measurement container 101 using one or more types of resin especially selected from polypropylene, polystyrene, acrylic, and polysulfone among them. The resins have a property of low coagulation activity with respect to a blood sample, so that they are suitable for measuring the blood sample.

Note that, in the present technology, a well-known disposable cartridge type may also be used as the electrical measurement container 101.

The present technology is preferably provided with one or a plurality of electrical measurement containers including an assay that at least evaluates extrinsic coagulation ability. Therefore, a TFPI may be efficiently evaluated by the analysis unit 4 to be described later. Examples of the assay include that containing a tissue factor and calcium as reagents and the like, for example, and it is preferable that these reagents are sealed in advance in one or a plurality of electrical measurement containers.

In the present technology, in a case where a drug is used in this manner, it is also possible to store a predetermined drug in advance in the electrical measurement container 101 as a solid or as a liquid. For example, an anticoagulant, a coagulation initiator, a tissue factor, a heparin decomposing agent, a heparin neutralizing agent, an anti-TFPI antibody and the like may be stored in the container 101 in advance. By storing the drug in the container 101 in advance in this manner, the drug supply unit 11 to be described later and a portion for holding the drug are not required, and the device may be made compact and a cost may be reduced. Furthermore, usability may be improved because a user does not have to replace the drug and device maintenance of the drug supply unit 11, the portion for holding the drug and the like is not required.

(1-2) Connection Unit 102

A connection unit 102 electrically connects the application unit 3 to be described later to the electrodes 1a and 1b. A specific form of the connection unit 102 is not especially limited, and this may be freely designed as appropriate as long as the application unit 3 and the electrodes 1a and 1b may be electrically connected to each other.

(1-3) Container Holding Unit 103

A container holding unit 103 holds the electrical measurement container 101. A specific form of the container holding unit 103 is not especially limited, and this may be freely designed as appropriate as long as the container 101 in which the blood sample B to be analyzed is stored may be held.

A material forming the container holding unit 103 is not especially limited, too, and this may be freely selected as appropriate according to the form and the like of the electrical measurement container 101.

Furthermore, in the present technology, the container holding unit 103 may have a function of automatically reading information regarding the container 101 from an information recording medium provided on the electrical measurement container 101 (for example, a bar code reader). Examples of the information storage medium include, for example, an IC card, an IC tag, a card provided with a bar code or a matrix-type two-dimensional code, paper or a sticker on which the bar code or the matrix-type two-dimensional code is printed and the like.

(2) Application Unit 2

The application unit 2 applies an alternating voltage to the pair of electrodes 1a and 1b at a predetermined time interval. More specifically, for example, the application unit 2 applies the alternating voltage to the pair of electrodes 1a and 1b from a time point when a command to start the measurement is received or a time point when the device 10 is powered on as a starting point. More specifically, the application unit 2 applies the alternating voltage at a set frequency or a frequency controlled by the measurement condition control unit 8 to be described later to the pair of electrodes 1a and 1b at a set measurement interval or a measurement interval controlled by the measurement condition control unit 8 to be described later.

(3) Measurement Unit 3

The measurement unit 3 measures the complex permittivity of the blood sample arranged between the pair of electrodes 1a and 1b. A configuration of the measurement unit 3 may be freely designed as appropriate as long as it is configured such that the complex permittivity, which is a measurement target, of the blood sample B may be measured. Specifically, for example, an impedance analyzer, a network analyzer and the like may be adopted as the measurement unit 3.

More specifically, for example, it is configured to measure impedance of the blood sample B obtained by application of the alternating voltage to the blood sample B by the application unit 2 over time, and a configuration that the impedance of the blood sample B between the electrodes 1a and 1b is measured over time from the time point when the command to start the measurement is received or the time point when the device 10 is powered on as the starting point may be adopted. Then, the complex permittivity is derived from the measured impedance. It is possible to use well-known function and relational expression indicating a relationship between the impedance and the permittivity for deriving the complex permittivity.

A measurement result by the measurement unit 3 may be obtained as a three-dimensional complex permittivity spectrum (FIG. 2) with the frequency, time, and permittivity plotted along coordinate axes, respectively, or a two-dimensional complex permittivity spectrum (FIG. 3) with two selected out of the frequency, time, and permittivity plotted along coordinate axes, respectively. A real part of the complex permittivity at each time and each frequency is plotted along the Z-axis in FIG. 2.

FIG. 3 corresponds to the two-dimensional spectrum obtained by cutting out the three-dimensional spectrum illustrated in FIG. 2 at a frequency of 760 kHz. In FIG. 3, reference sign (A) indicates a peak associated with rouleaux formation of erythrocytes, and reference sign (B) indicates a peak associated with a blood sample coagulation process. The inventors of the present application have clarified in Patent Document 1 described above that a change in time in permittivity of the blood sample reflects the coagulation process of the blood sample. Therefore, the complex permittivity spectrum obtained by the measurement unit 3 is an index that quantitatively indicates coagulation ability of the blood sample, and on the basis of change thereof, it is possible to obtain information regarding the coagulation ability of the blood sample such as a blood sample coagulation time, a blood sample coagulation speed, and a blood sample coagulation strength.

(4) Analysis Unit 4

The analysis unit 4 evaluates a human tissue factor pathway inhibitor (TFPI) on the basis of the complex permittivity at a specific frequency in a predetermined period measured at the time interval after anticoagulant action acting on the blood sample is released.

Specifically, the analysis unit 4 evaluates the TFPI by using, for example, a tissue factor (TF) and an anti-TFPI antibody.

More specifically, the complex permittivity measured by using the tissue factor and the anti-TFPI antibody is compared with the complex permittivity measured by using the tissue factor, and the TFPI is evaluated on the basis of a difference between the spectral patterns. The spectral patterns may be compared on the basis of a feature amount in the change of the complex permittivity at the specific frequency of both, and the difference between the spectral patterns may be detected from the difference in the feature amount. As the feature amount, a temporal index related to a blood sample coagulation reaction, an index related to a speed of the reaction and the like may be adopted.

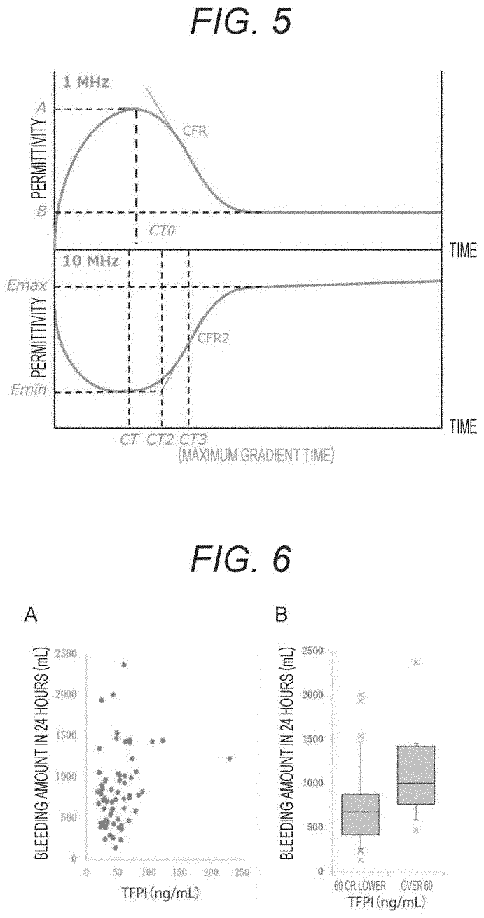

FIG. 5 is a drawing-substituting graph illustrating an example of the feature amount extracted from the complex permittivity spectrum. In FIG. 5, the permittivity and time are plotted along the ordinate and abscissa, respectively, an upper graph is based on the measurement result at a frequency around 1 MHz (100 kHz or higher and lower than 3 MHz), and a lower graph is based on the measurement result at a frequency around 10 MHz (3 to 30 MHz).

In the present technology, as the feature amount, a time feature amount and/or a gradient feature amount extracted from the complex permittivity spectrum at the specific frequency may be used. Furthermore, the gradient feature amount may be extracted on the basis of the time feature amount extracted from the complex permittivity spectrum at the specific frequency. More specifically, as the feature amount, for example, any one or more selected from a group including time CT0 at which a local maximum value of the complex permittivity is given at a low frequency of 100 kHz or higher and lower than 3 MHz, time CT1 (not illustrated) at which a maximum gradient is given at the low frequency, a maximum gradient CFR at the low frequency, time CT4 (not illustrated) when an absolute value of the gradient reaches a predetermined percentage (preferably, 50%) of the CFR after the CT1, time CT at which a local minimum value of the complex permittivity is given at a high frequency of 3 to 30 MHz, time CT3 at which a maximum gradient is given at the high frequency, a maximum gradient CFR2 at the high frequency, time CT2 at which an absolute minimum value of the complex permittivity is given when a straight line is drawn at the gradient of CFR2 from CT3 after the CT and before the CT3, and time CT5 (not illustrated) when an absolute value of the gradient reaches a predetermined percentage (preferably, 50%) of the CFR2 after the CT3. Furthermore, it is also possible to use a calculated value of the feature amounts and a calculated value with the measured complex permittivity and the like.

More specifically, if the blood coagulation time (for example, CT0 and the like) measured by using the tissue factor and anti-TFPI antibody is shorter than the blood coagulation time measured by using the tissue factor, this shortening is obtained by inhibiting the TFPI in a specimen (blood sample B) with the anti-TFPI antibody, so that it may be evaluated that a blood concentration of the TFPI increases in such specimen.

When the blood concentration of the TFPI increases, there is a possibility that, even in a blood vessel damaged site where blood coagulation reaction should originally occur, the reaction is inhibited, and effective hemostasis cannot be performed. Therefore, it is also possible to analyze, for example, a degree of postoperative bleeding risk by determining whether or not the TFPI concentration in the blood is high.

Furthermore, the inventors of the present application have clarified that the TFPI concentration in the blood affect a postoperative bleeding amount in examples to be described later. Therefore, as the postoperative bleeding risk, for example, the bleeding amount may also be predicted by the analysis unit 4. Note that in a case of the specimen in which the blood coagulation time of a case measured by using the tissue factor and anti-TFPI antibody is shorter than the blood coagulation time of a case measured by using the tissue factor described above, it may be determined that this specimen originally has a high bleeding risk due to the TFPI, so that the bleeding risk may be reduced by using the anti-TFPI antibody.

In the present technology, it is further preferable to evaluate the TFPI by using the heparin decomposing agent and/or the heparin neutralizing agent. By using them, even the specimen containing residual heparin may be evaluated excluding anticoagulant action of heparin. Examples of the heparin decomposing agent include heparinase and the like, for example, and examples of the heparin neutralizing agent include protamine, polybrene and the like, for example.

In the present technology, it is more preferable to evaluate the TFPI by especially using the heparin decomposing agent among them. This is because, in a case of the heparin decomposing agent, there is no possibility that this affects the measurement result even if this is excessively added, and a stable measurement result may be obtained.

In a case of evaluating the TFPI by using the heparin decomposing agent and/or the heparin neutralizing agent, more specifically, the complex permittivity measured by using the tissue factor and anti-TFPI antibody is compared with the complex permittivity measured by using the tissue factor, and the TFPI is evaluated on the basis of the difference between the spectral patterns. Since a method of evaluating the TFPI on the basis of the difference between the spectral patterns is similar to that described above, the description thereof is herein omitted.

(5) Notification Unit 5

The notification unit 5 performs notification of the analysis result by the analysis unit 4 at a specific time point. In the present technology, a configuration of the notification unit 5 is not especially limited, and for example, it may be configured to generate a notification signal only in a case where an abnormal analysis result is obtained during the measurement and notify the user of the result in real time. Therefore, the user is notified of the analysis result only at a specific time point when the abnormal analysis result is confirmed, so that usability is improved.

Furthermore, a method of notifying the user is not especially limited, and for example, the notification may be performed via the display unit 6 to be described later, a display, a printer, a speaker, lighting and the like. Furthermore, for example, a device having a communication function for sending an e-mail and the like for notifying that the notification signal is generated to a mobile device such as a mobile phone, a smartphone and the like may also be used as the notification unit 5.

Furthermore, in the present technology, the notification unit 5 may have, for example, a function of notifying the user of a warning and the like to urge the user to set the container 101 in a case where one or a plurality of electrical measurement containers 101 including the assay that at least evaluates the extrinsic coagulation ability described above is not set in the device 100 even though it is input in advance to the device 100 that the TFPI is evaluated.

(6) Display Unit 6

The display unit 6 displays the analysis result by the analysis unit 4, data of the complex permittivity measured by the measurement unit 3, the notification result from the notification unit 5 and the like. A configuration of the display unit 6 is not especially limited, and for example, a display, a printer and the like may be adopted as the display unit 6. Furthermore, in the present technology, the display unit 6 is not indispensable, and an external display device may be connected.

(7) Storage Unit 7

The storage unit 7 stores the analysis result by the analysis unit 4, the data of the complex permittivity measured by the measurement unit 3, the notification result from the notification unit 5 and the like. A configuration of the storage unit 7 is not especially limited, and for example, as the storage unit 7, a hard disk drive, a flash memory, a solid state drive (SSD) and the like may be adopted, for example. Furthermore, in the present technology, the storage unit 7 is not indispensable, and an external storage device may be connected.

Moreover, in the present technology, an operation program and the like of the blood coagulation system analysis device 100 may be stored in the storage unit 7.

(8) Measurement Condition Control Unit 8

The measurement condition control unit 8 controls a measurement time and/or a measurement frequency and the like in the measurement unit 3. As a specific method of controlling the measurement time, a measurement interval may be controlled according to an amount of data required for analyzing a target and the like, or a timing of finishing the measurement may be controlled in a case where a measured value becomes almost flat and the like.

Furthermore, it is also possible to control the measurement frequency according to a type of the blood sample B to be measured, the measured value required for analyzing the target and the like. Control of the measurement frequency includes a method of changing the frequency of the alternating voltage applied between the electrodes 1a and 1b, a method of superimposing a plurality of frequencies and performing impedance measurement at a plurality of frequencies and the like. Specifically, as a specific method thereof, there may be a method of arranging a plurality of single-frequency analyzers in parallel, a method of sweeping frequencies, a method of superimposing frequencies and extracting information of each frequency with a filter, a method of measuring with a response to an impulse and the like.

(9) Temperature Control Unit 9

The temperature control unit 9 controls temperature in the electrical measurement container 101. In the blood coagulation system analysis device 100 according to the present technology, the temperature control unit 9 is not indispensable, but it is preferable to provide the same in order to maintain the blood sample B to be analyzed in an optimal state for measurement.

Furthermore, as described later, in a case where the sample standby unit 14 is provided, the temperature control unit 9 may also control the temperature in the sample standby unit 14. Moreover, in a case where a drug is put into the blood sample B at the time of measurement or before the measurement, the temperature control unit 9 may be provided in order to control temperature of the drug. In this case, the temperature control unit 9 may be provided for each of temperature control in the electrical measurement container 101, temperature control in the sample standby unit 14, and temperature control of the drug, or one temperature control unit 9 may control the temperature of all of them.

A specific method of the temperature control is not especially limited, but for example, it is possible to allow the container holding unit 103 to serve as the temperature control unit 9 by giving a temperature adjusting function to the container holding unit 103.

(10) Blood Sample Supply Unit 10

The blood sample supply unit 10 automatically supplies the blood sample B to the electrical measurement container 101. In the blood coagulation system analysis device 100 according to the present technology, the blood sample supply unit 10 is not indispensable, but by providing the blood sample supply unit 8, each step of the blood coagulation system analysis may be automatically performed.

A specific method of supplying the blood sample B is not especially limited, but for example, the blood sample B may be automatically supplied to the electrical measurement container 101 by using a pipettor and a tip attached to a tip end thereof. In this case, it is preferable that the tip is disposable in order to prevent a measurement error and the like. Furthermore, the blood sample B may be automatically supplied from a storage of the blood sample B to the electrical measurement container 101 by using a pump and the like. Moreover, it is also possible to automatically supply the blood sample B to the electrical measurement container 101 by using a permanently installed nozzle and the like. In this case, it is preferable to give a cleaning function to the nozzle in order to prevent the measurement error and the like.

Furthermore, in the present technology, it is also possible to give a function of identifying the type and the like of the blood sample B as the specimen and automatically reading the same (for example, a barcode reader and the like) to the blood sample supply unit 10.

(11) Drug Supply Unit 11

The drug supply unit 11 automatically supplies one or two or more types of drugs to the electrical measurement container 101. In the blood coagulation system analysis device 100 according to the present technology, the drug supply unit 11 is not indispensable, but by providing the drug supply unit 11, each step of blood coagulation system analysis may be automatically performed.

A specific method of supplying the drug is not especially limited, and this may be supplied by using a method similar to that of the blood sample supply unit 10 described above. Especially, a method capable of supplying a constant amount of drug without contact with the electrical measurement container 101 is preferable when supplying the drug. For example, a liquid drug may be supplied by discharging. More specifically, for example, it is possible to introduce a drug solution into a discharge pipe in advance, and, via a pipeline connected to the same, blow separately connected pressurized air into the pipeline for a short time, thereby discharging to supply the drug solution to the container 101. At that time, a discharge amount of the drug solution may be adjusted by adjusting an air pressure and a valve opening/closing time.

Furthermore, in addition to blowing air, it is also possible to discharge to supply the drug solution to the container 101 by utilizing vaporization of the drug solution itself or air dissolved therein by heating. At that time, it is possible to adjust the discharge amount of the drug solution by adjusting a volume of generated bubbles by adjusting an applied voltage to a vaporization chamber in which a heat generating element and the like is installed and a time thereof.

Moreover, it is also possible to supply the drug solution to the container 101 not by using air but by using a piezoelectric element (piezo element) and the like to drive a movable unit provided in the pipeline and deliver the drug solution of an amount determined by a volume of the movable unit. Furthermore, for example, it is also possible to supply the drug by using a so-called inkjet system in which the drug solution is atomized and directly sprayed onto the desired container 101.

Furthermore, in the present technology, it is also possible to give a stirring function, a temperature controlling function, and a function of identifying the type and the like of the drug and automatically reading the same (for example, the barcode reader) to the drug supply unit 11.

(12) Accuracy Management Unit 12

The accuracy management unit 12 manages accuracy of the measurement unit 3. In the blood coagulation system analysis device 100 according to the present technology, this accuracy management unit 12 is not indispensable, but by providing the accuracy management unit 12, it is possible to improve measurement accuracy in the measurement unit 3 and improve usability.

A specific accuracy managing method is not especially limited, and a well-known accuracy managing method may be freely used as appropriate. There may be a method of managing accuracy of the measurement unit 3 and the like by calibrating the measurement unit 3: for example, a method of calibrating the measurement unit 3 by installing a metal plate and the like for short-circuiting in the device 100 and short-circuiting an electrode and the metal plate before starting the measurement, a method of bringing a jig for calibration and the like into contact with the electrode, a method of calibrating the measurement unit 3 by installing a metal plate and the like in a container having the same form as that of the container 101 in which the blood sample B is put and short-circuiting the electrode and the metal plate before starting the measurement and the like.

Furthermore, in addition to the method described above, it is possible to select a free method to use as appropriate such as a method of checking the state of the measurement unit 3 before actual measurement and performing the above-described calibration and the like only when there is an abnormality to calibrate the measurement unit 3, thereby managing the accuracy of the measurement unit 3.

(13) Driving Mechanism 13

The driving mechanism 13 is used to move the electrical measurement container 101 in the measurement unit 3 according to various purposes. For example, by moving the container 101 in a direction to change a direction of gravity applied to the blood sample B held in the container 101, it is possible to prevent an influence on a measured value by sedimentation of a sedimentation component in the blood sample B.

Furthermore, for example, it is also possible to drive the electrical measurement container 101 such that the application unit 2 and the electrodes 1a and 1b are put into a disconnected state at the time of non-measurement, and the application unit 2 may be electrically connected to the electrodes 1a and 1b at the time of measurement.

Moreover, for example, in a case where a plurality of electrical measurement containers 101 is provided, if it is configured such that the containers 101 are movable, it is possible to measure, supply the blood sample, and supply the drug by moving the container 101 to a required site. That is, since it is not required to move the measurement unit 3, the blood sample supply unit 10, the drug supply unit 11 and the like to a target electrical measurement container 101, it is not required to provide a drive unit and the like for moving each unit and the device may be made compact and a cost may be reduced.

(14) Sample Standby Unit 14

The sample standby unit 14 allows a separated blood sample B to stand by before the measurement. In the blood coagulation system analysis device 100 according to the present technology, the sample standby unit 12 is not indispensable, but by providing the sample standby unit 14, the permittivity may be smoothly measured.

In the present technology, it is also possible to give a stirring function, a temperature controlling function, a moving mechanism to the electrical measurement container 101, a function of identifying the type and the like of the blood sample B and automatically reading the same (for example, the barcode reader and the like), an automatic opening function and the like to the sample standby unit 14.

(15) Stirring Mechanism 15

The stirring mechanism 15 stirs the blood sample B and stirs the blood sample B and the drug. In the blood coagulation system analysis device 100 according to the present technology, the stirring mechanism 13 is not indispensable, but for example, in a case where the blood sample B contains a sedimentation component or in a case where a drug is added to the blood sample B at the time of measurement, it is preferable that the stirring mechanism 15 is provided.

A specific stirring method is not especially limited, and a well-known stirring method may be freely used as appropriate. For example, there may be stirring by pipetting, stirring using a stirring rod, a stirrer and the like, stirring by turning the container containing the blood sample B and the drug upside down and the like.

(16) User Interface 16

The user interface 16 is a portion for the user to operate. The user may access each unit of the blood coagulation system analysis device 100 via the user interface 16.

(17) Server 17

The server 17 is at least provided with a storage unit that stores data in the measurement unit 3 and/or the analysis result by the analysis unit 4, and is connected to at least the measurement unit 3 and/or the analysis unit 4 via a network.

Furthermore, the server 17 may manage various data uploaded from each unit of the blood coagulation system analysis device 100 and output the various data to the display unit 6 and the like according to an instruction from the user.

(18) Others

Note that it is also possible to store a function performed in each unit of the blood coagulation system analysis device 100 according to the present technology in a personal computer and a hardware resource provided with a control unit including a CPU and the like, a recording medium (non-volatile memory (such as USB memory), HDD, CD and the like) and the like as a program, and allow the same to serve by the personal computer and the control unit.

### Examples

Hereinafter, the present technology is described in further detail on the basis of an example.

Note that the example hereinafter described illustrates an example of a representative example of the present invention, and the scope of the present technology is not narrowed by them.

<Specimen>

Measurements by blood were performed on adult patients undergoing cardiovascular surgery using an artificial heart-lung machine. Timings of blood collection were as follows.

(i) After introduction of anesthesia and before start of surgery

(ii) After artificial heart-lung machine is finished and at end of heparin neutralization with protamine

(iii) One hour after (ii)

(iv) Two hours after (ii) (in case where chest is already closed at that time point, proceed to (v))

(v) At end of surgery after chest closure

<Measurement>

In addition to measurement by a blood coagulation system analysis device, blood count, general coagulation test, measurement of coagulation/fibrinolysis/adjusting factors including TFPI using plasma were performed. Furthermore, a bleeding amount from a drain after surgery was measured. Moreover, in the measurement by the blood coagulation system analysis device, the measurement was also carried out for that to which an anti-TFPI antibody was added, and comparison and examination with a control to which the antibody on the left was not added was also carried out.

In the blood coagulation system analysis device, a blood collection tube in which blood was collected using citric acid as an anticoagulant was set in a blood sample supply unit of the device, and automatically heated to 37° C. by a temperature control unit. Note that specimen information may be input via a user interface or may be automatically input by reading of a barcode.

An electrical measurement container filled with a reagent in advance was set in a measurement unit controlled at 37° C. Note that the reagent in the electrical measurement container is different for each assay, and it is possible to simultaneously measure by using a plurality of electrical measurement containers (assays). A user may input the fact to give priority to evaluation of TFPI and other items via the user interface, or allow to automatically read the same via an information storage medium such as a bar code attachable to the electrical measurement container and the like.

In order to evaluate the TFPI, it is preferable that the assay capable of at least evaluating extrinsic coagulation ability (for example, that containing a tissue factor and calcium as reagents) is set in the device. For convenience, this assay is referred to as “EX” in this example. Furthermore, in order to perform evaluation excluding an effect of heparin, the assay obtained by adding heparinase to EX is referred to as “EXHN” in this example for convenience. Furthermore, that obtained by adding an anti-TFPI antibody to this EXHN is referred to as “EXHNT” for convenience in this example.

<Result>

A and B of FIG. 6 are drawing-substituting graphs illustrating a relationship between a TFPI concentration in plasma and a bleeding amount within 24 hours after the surgery obtained in a measurement group examined this time. In these graphs, the bleeding amount (mL) within 24 hours and the TFPI concentration in plasma (ng/mL) are plotted along the ordinate and the abscissa, respectively. From this result, it was illustrated that when a TFPI value was high, postoperative bleeding increased significantly, and it was found that the TFPI concentration in the blood affected the postoperative bleeding amount.

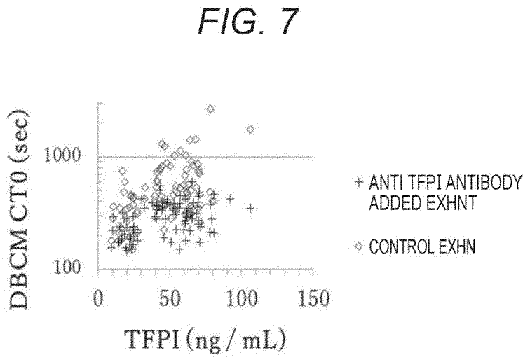

FIG. 7 is a drawing-substituting graph comparing results of EXHNT and EXHN while focusing on CT0 (=time at which a local maximum value of complex permittivity is given at a low frequency of 100 kHz or higher and lower than 3 MHz, here, blood coagulation time) from an analysis result of the blood coagulation system analysis device in the measurement group examined this time. The CT0 (sec) and the TFPI concentration in plasma (ng/mL) are plotted along the ordinate and abscissa, respectively. As is clear from this result, it may be understood that as the TFPI concentration increases, the CT0 is prolonged in the EXHN and blood coagulation ability (hemostatic ability) is decreased. This is associated with the increase in postoperative bleeding as the TFPI concentration becomes higher, as illustrated in FIG. 6.

On the other hand, in the EXHNT assay to which the anti-TFPI antibody was added, the prolongation of the CT0 is inhibited even in a specimen with high TFPI concentration, and the coagulation ability is maintained. Since it may be understood that the decrease in coagulation ability is due to the TFPI in such specimen, it may be presented as a test result that the postoperative bleeding may be inhibited by treatment with the anti-TFPI antibody.

From the above, according to the present technology, it is possible to evaluate a degree of blood coagulation inhibiting effect by the TFPI regarding the TFPI in blood, which is one of causes of the postoperative bleeding. Furthermore, since the TFPI inhibiting effect by the anti-TFPI antibody may be understood, it is possible to distinguish between a patient group in which the anti-TFPI antibody drug is effective and a patient group in which the anti-TFPI antibody drug is not effective, and it is possible to determine whether an effect of the TFPI is large or another factor is large as a risk of the postoperative bleeding and contribute to determination of an optimal treatment policy for each patient.

Note that the present technology may also take the following configuration.

(1)

A blood coagulation system analysis device provided with:

a pair of electrodes;

an application unit that applies an alternating voltage to the pair of electrodes at a predetermined time interval;

a measurement unit that measures complex permittivity of a blood sample arranged between the pair of electrodes; and

an analysis unit that evaluates a human tissue factor pathway inhibitor (TFPI) on the basis of the complex permittivity at a specific frequency in a predetermined period measured at the time interval after anticoagulant action acting on the blood sample is released.

(2)

The blood coagulation system analysis device according to (1), in which the TFPI is evaluated by using a tissue factor and an anti-TFPI antibody.

(3)

The blood coagulation system analysis device according to (2), in which the analysis unit evaluates the TFPI on the basis of the complex permittivity measured by using the tissue factor and the anti-TFPI antibody and the complex permittivity measured by using the tissue factor.

(4)

The blood coagulation system analysis device according to (2) or (3), in which a heparin decomposing agent and/or a heparin neutralizing agent are further used.

(5)

The blood coagulation system analysis device according to (4), in which the analysis unit evaluates the TFPI on the basis of the complex permittivity measured by using the tissue factor, the heparin decomposing agent and/or the heparin neutralizing agent, and the anti-TFPI antibody, and the complex permittivity measured by using the tissue factor and the heparin decomposing agent and/or the heparin neutralizing agent.

(6)

The blood coagulation system analysis device according to any one of (1) to (5), in which a feature amount extracted from a complex permittivity spectrum at the specific frequency is used at the time of the evaluation.

(7)

The blood coagulation system analysis device according to (6), in which the feature amount is a time feature amount and/or a gradient feature amount extracted from the complex permittivity spectrum at the specific frequency.

(8)

The blood coagulation system analysis device according to (7), in which the gradient feature amount is extracted on the basis of the time feature amount extracted from the complex permittivity spectrum at the specific frequency.

(9)

The blood coagulation system analysis device according to any one of (6) to (8), in which the feature amount is any one or more selected from a group including time CT0 at which a local maximum value of the complex permittivity is given at a low frequency of 100 kHz or higher and lower than 3 MHz, time CT1 at which a maximum gradient is given at the low frequency, a maximum gradient CFR at the low frequency, time CT4 when an absolute value of the gradient reaches a predetermined percentage of the CFR after the CT1, time CT at which a local minimum value of the complex permittivity is given at a high frequency of 3 to 30 MHz, time CT3 at which a maximum gradient is given at the high frequency, a maximum gradient CFR2 at the high frequency, time CT2 at which an absolute minimum value of the complex permittivity is given when a straight line is drawn at the gradient of CFR2 from CT3 after the CT and before the CT3, and time CT5 when an absolute value of the gradient reaches a predetermined percentage of the CFR2 after the CT3.

(10)

The blood coagulation system analysis device according to any one of (1) to (9), in which the analysis unit analyzes a degree of postoperative bleeding risk.

(11)

The blood coagulation system analysis device according to (10), in which the bleeding risk is a bleeding amount.

(12)

The blood coagulation system analysis device according to any one of (1) to (11), further provided with:

one or a plurality of electrical measurement containers including an assay that at least evaluates extrinsic coagulation ability.

### REFERENCE SIGNS LIST

100 Blood coagulation system analysis device
1a, 1b Pair of electrodes
101 Electrical measurement container
102 Connection unit
103 Container holding unit
2 Application unit
3 Measurement unit
4 Analysis unit
5 Notification unit
6 Display unit
7 Storage unit
8 Measurement condition control unit
9 Temperature control unit
10 Blood sample supply unit
11 Drug supply unit
12 Accuracy control unit
13 Driving mechanism
14 Sample standby unit
15 Stirring mechanism
16 User interface
17 Server

1. A blood coagulation system analysis device comprising:
a pair of electrodes;
an application unit that applies an alternating voltage to the pair of electrodes at a predetermined time interval;
a measurement unit that measures complex permittivity of a blood sample arranged between the pair of electrodes; and
an analysis unit that evaluates a human tissue factor pathway inhibitor (TFPI) on a basis of the complex permittivity at a specific frequency in a predetermined period measured at the time interval after anticoagulant action acting on the blood sample is released.

2. The blood coagulation system analysis device according to claim 1, wherein the TFPI is evaluated by using a tissue factor and an anti-TFPI antibody.

3. The blood coagulation system analysis device according to claim 2, wherein the analysis unit evaluates the TFPI on a basis of the complex permittivity measured by using the tissue factor and the anti-TFPI antibody and the complex permittivity measured by using the tissue factor.

4. The blood coagulation system analysis device according to claim 2, wherein a heparin decomposing agent and/or a heparin neutralizing agent are further used.

5. The blood coagulation system analysis device according to claim 4, wherein the analysis unit evaluates the TFPI on a basis of the complex permittivity measured by using the tissue factor, the heparin decomposing agent and/or the heparin neutralizing agent, and the anti-TFPI antibody, and the complex permittivity measured by using the tissue factor and the heparin decomposing agent and/or the heparin neutralizing agent.

6. The blood coagulation system analysis device according to claim 1, wherein a feature amount extracted from a complex permittivity spectrum at the specific frequency is used at the time of the evaluation.

7. The blood coagulation system analysis device according to claim 6, wherein the feature amount is a time feature amount and/or a gradient feature amount extracted from the complex permittivity spectrum at the specific frequency.

8. The blood coagulation system analysis device according to claim 7, wherein the gradient feature amount is extracted on a basis of the time feature amount extracted from the complex permittivity spectrum at the specific frequency.

9. The blood coagulation system analysis device according to claim 6, wherein the feature amount is any one or more selected from a group including time CT0 at which a local maximum value of the complex permittivity is given at a low frequency of 100 kHz or higher and lower than 3 MHz, time CT1 at which a maximum gradient is given at the low frequency, a maximum gradient CFR at the low frequency, time CT4 when an absolute value of the gradient reaches a predetermined percentage of the CFR after the CT1, time CT at which a local minimum value of the complex permittivity is given at a high frequency of 3 to 30 MHz, time CT3 at which a maximum gradient is given at the high frequency, a maximum gradient CFR2 at the high frequency, time CT2 at which an absolute minimum value of the complex permittivity is given when a straight line is drawn at the gradient of CFR2 from CT3 after the CT and before the CT3, and time CT5 when an absolute value of the gradient reaches a predetermined percentage of the CFR2 after the CT3.

10. The blood coagulation system analysis device according to claim 1, wherein the analysis unit analyzes a degree of postoperative bleeding risk.

11. The blood coagulation system analysis device according to claim 10, wherein the bleeding risk is a bleeding amount.

12. The blood coagulation system analysis device according to claim 1, further comprising:
one or a plurality of electrical measurement containers including an assay that at least evaluates extrinsic coagulation ability.
